(19) United States
(12) Patent Application Publication
(10) Pub. No.: US 2005/0175667 A1
(43) Pub. Date: Aug. 11, 2005

(54) Use of endothelin antagonists to prevent restenosis

(76) Inventor: Wenda Carlyle, Silverado, CA (US)

Correspondence Address: EDWARDS LIFESCIENCES CORPORATION, ONE EDWARDS WAY, LEGAL DEPARTMENT, IRVINE, CA, 92614, US

(21) Appl. No.: 11/054,009

(22) Filed: Feb. 8, 2005

Related U.S. Application Data
(60) Provisional application No. 60/543,252, filed on Feb. 10, 2004.

(51) Int. Cl. A61F 2/00; A61M 31/00; A61K 31/137

(52) U.S. Cl. 424/423; 604/500; 514/649

(57) ABSTRACT

Provided are devices and methods for treating or preventing smooth muscle cell proliferation caused by endothelin-mediated conditions. In particular, a medical device comprising a structure which is implantable within a body lumen and means on or within the structure for releasing an endothelin (A) receptor antagonist at a rate effective to inhibit smooth muscle cell proliferation. The device can be, for example, an expansible stent or a graft, and the means can include a matrix coating, wherein the endothelin (A) receptor antagonist can be dispersed within the coating or disposed directly on the structure and under the matrix. The methods and devices of this invention can be used to decrease the incidence of restenosis as well as other thromboembolic complications resulting from implantation of medical devices.

### CROSS REFERENCE TO A RELATED PATENT APPLICATION

Priority is herewith claimed under 35 U.S.C. §119(e) from co-pending Provisional Patent Application No.: 60/543,252, filed Feb. 10, 2004, entitled “USE OF ENDOTHELIN ANTAGONISTS TO PREVENT RESTENOSIS”. The disclosure of this Provisional Patent Application is incorporated by reference herein in its entirety.

### BACKGROUND OF THE INVENTION

1. Field of the Invention

The present invention relates to the field of intracorporeal medical devices that incorporate an endothelin (A) receptor antagonist. One particular aspect of this invention provides improved devices and methods for minimizing and/or inhibiting restenosis and hyperplasia after intravascular intervention.

2. Description of the Prior Art

Coronary artery atherosclerosis disease (CAD) is the most common, serious, chronic, life-threatening illness in the United States, affecting more than 11 million persons. Atherosclerosis involves the deposition of fatty plaques on the lumenal surface of arteries, which in turn causes narrowing of the cross-sectional area of the artery. Ultimately, this deposition blocks blood flow distal to the lesion causing ischemic damage to the tissues supplied by the artery. The social and economic costs of coronary atherosclerosis vastly exceed that of most other diseases. Narrowing of the coronary artery lumen causes destruction of heart muscle resulting first in angina, followed by myocardial infarction and finally death. There are over 1.5 million myocardial infarctions in the United States each year, and six hundred thousand (or 40%) of those patients suffer an acute myocardial infarction and more than three hundred thousand of those patients die before reaching the hospital (Harrison's Principles of Internal Medicine, 14th Edition, 1998).

A number of percutaneous intravascular procedures have been developed for treating stenotic atherosclerotic regions of a patient's vasculature to restore adequate blood flow. The most successful of these treatments is percutaneous transluminal angioplasty (PTA). In PTA, a catheter, having an expansible distal end usually in the form of an inflatable balloon is inserted into a peripheral artery and threaded through the arterial system into the blocked coronary artery and is positioned in the blood vessel at the stenotic site. The balloon is then inflated to flatten the obstructing fatty plaque and dilate the vessel, thereby restoring adequate blood flow beyond the diseased region. Other procedures for opening stenotic regions include directional arthrectomy, rotational arthrectomy, laser angioplasty, stenting, and the like. While these procedures have gained wide acceptance (either alone or in combination, such as PTA in combination with stenting), they continue to suffer from significant disadvantages. A particularly common disadvantage with PTA and other known procedures for opening stenotic regions is the frequent occurrence of restenosis.

Restenosis refers to the re-narrowing of an artery after an initially successful angioplasty. Restenosis afflicts approximately up to 50% of all angioplasty patients and is the result of injury to the blood vessel wall during the lumen opening angioplasty procedure. In some patients, the injury initiates a repair response that is characterized by smooth muscle cell proliferation referred to as “hyperplasia” in the region traumatized by the angioplasty. Acutely, restenosis involves recoil and shrinkage of the vessel, which are followed by proliferation of medial smooth muscle cells. This proliferation of smooth muscle cells re-narrows the lumen that was opened by the angioplasty within a few weeks to a few months, thereby necessitating a repeat PTA or other procedure to alleviate the restenosis. As many as 50% of the patients who are treated by PTCA require a repeat procedure within six months to correct restenosis.

Narrowing of the arteries. can occur in vessels other than the coronary arteries, including, but not limited to, the aortoiliac, infrainguinal, distal profunda femoris, distal popliteal, tibial, subclavian, mesenteric, carotid, and renal arteries. Peripheral artery atherosclerosis disease (“PAD”, also known as peripheral arterial occlusive disease) commonly occurs in arteries in the extremities (feet, hands, legs, and arms). Rates of PAD appear to vary with age, with an increasing incidence of PAD in older individuals. Data from the National Hospital Discharge Survey estimate that every year, 55,000 men and 44,000 women have a first-listed diagnosis of chronic PAD and 60,000 men and 50,000 women have a first-listed diagnosis of acute PAD. Ninety-one percent of the acute PAD cases involved the lower extremity. The prevalence of comorbid CAD in patients with PAD can exceed 50%. In addition, there is an increased prevalence of cerebrovascular disease among patients with PAD.

PAD can be treated using percutaneous translumenal balloon angioplasty (PTA). Preferably, PAD is treated using bypass procedures where the blocked section of the artery is bypassed using a graft (Principles of Surgery, Schwartz et al. eds., Chapter 20, Arterial Disease, 7th Edition, McGraw-Hill Health Professions Division, New York 1999). The graft can consist of an autologous venous segment such as the saphenous vein or a synthetic graft such as one made of polyester, polytetrafluoroethylene (PTFE), or expanded polytetrafluoroethylene (ePTFE). The post-operative patency rates depend on a number of different factors, including the lumenal dimensions of the bypass graft, the type of synthetic material used for the graft and the site of outflow. Restenosis and thrombosis, however, remain significant problems even with the use of bypass grafts.

A number of different techniques have been used to overcome the problem of restenosis, including treatment of patients with various pharmacological agents or mechanically holding the artery open with a stent or synthetic vascular graft (Harrison's Principles of Internal Medicine, 14th Edition, 1998). Of the various procedures used to overcome restenosis, stents have proven to be the most effective. Stents are metal scaffolds that are permanently implanted in the diseased vessel segment to hold the lumen open and improve blood flow. Placement of a stent in the affected arterial segment thus prevents recoil and subsequent closing of the artery. Stents can also prevent local dissection of the artery along the medial layer of the artery. By maintaining a larger lumen than that created using PTCA alone, stents reduce restenosis by as much as 30%. However, their use can be limited by various factors, including size and location of the blood vessel, a complicated or tortuous vessel pathway, etc. Also, even a vessel with a stent may eventually develop restenosis.

Consequently, there is a significant need to improve the performance of both stents and synthetic bypass grafts in order to further reduce the morbidity and mortality of CAD and PAD. With stents, the approach has been to coat the stents with various anti-thrombotic or anti-restenotic agents in order to reduce thrombosis and restenosis. For example, stents have been coated with chemical agents such as heparin or phosphorylcholine. Some studies suggest a possible short term reduction in thrombosis, however treatment with these agents appears to have no long-term effect on preventing restenosis. Additionally, heparin can bind a wide variety of growth factors, thereby increasing their concentration and potentially augmenting cell proliferation in that vicinity. Nonetheless, it is not feasible to load stents with sufficient therapeutically effective quantities of either heparin or phosphorylcholine to make treatment of restenosis in this manner practical.

As with stents, synthetic grafts have also been treated in a variety of ways to reduce postoperative restenosis and thrombosis (Bos, et al., Current Status Archives Physio. Biochem., 106:100-115 (1998)). For example, composites of polyurethane such as meshed polycarbonate urethane have been reported to reduce restenosis as compared with ePTFE grafts. The surface of the graft has also been modified using radiofrequency glow discharge to add polyterephalate to the ePTFE graft. Synthetic grafts have also been impregnated with biomolecules such as collagen. However, none of these approaches has significantly reduced the incidence of thrombosis or restenosis over an extended period of time.

Endothelin is an endothelium-derived vasoconstrictor peptide that is released from the vascular endothelium. It is the most potent vasopressor known to date and is produced by numerous cell types, including the cells of the endothelium, trachea, kidney and brain. Endothelin peptides exhibit numerous biological activities in vitro and in vivo. Endothelin provokes a strong and sustained vasoconstriction in vivo in rats and in isolated vascular smooth muscle preparations; it also provokes the release of eicosanoids and endothelium-derived relaxing factor (EDRF) from perfused vascular beds.

Two distinct endothelin receptors, designated ET(A) and ET(B), have been identified and DNA clones encoding each receptor have been isolated (Arai et al., Nature 348: 730-732 (1990); Sakurai et al., Nature 348:732-735 (1990)). ET(A) receptors appear to be selective for endothelin-1 and are predominant in cardiovascular tissues. ET(B) receptors are predominant in noncardiovascular tissues, including the central nervous system and kidney (Sakurai et al., supra). In addition, ET(A) receptors occur on vascular smooth muscle, are linked to vasoconstriction and have been associated with cardiovascular, renal and central nervous system diseases, whereas ET(B) receptors are located on the vascular endothelium, linked to vasodilation (Takayanagi et al., FEBS Lttrs. 282:103-106 (1991)) and have been associated with bronchoconstrictive disorders.

Recent clinical human data have shown a correlation between increased endothelin levels and numerous disease states. For example, elevated levels of endothelin have been measured in patients suffering from ischemic heart disease (Yasuda, et al., Amer. Heart J., 119:801-806 (1990), Ray, et al., Br. Heart J., 67:383-386 (1992)). Circulating and tissue endothelin immunoreactivity is increased more than twofold in patients with advanced atherosclerosis (Lerman, et al., New Engl. J. Med., 325:997-1001), and increased circulating endothelin levels were observed in patients who underwent percutaneous transluminal coronary angioplasty (PTCA) (Tahara, et al., Metab. Clin. Exp., 40:1235-1237 (1991); Sanjay, et al., Circulation, 84 (Suppl. 4:726) (1991), and in individuals with pulmonary hypertension (Miyauchi et al., Jpn. J. Pharmacol., 58:279P (1992); Stewart, et al., Ann. Internal Medicine, 114:464-469 (1991)).

Recently, a number of publications have described the use of endothelin receptor antagonists to prevent restenosis. It has been recognized that compounds that exhibit activity at IC₅₀ or EC₅₀ concentrations on the order of 10⁻⁴ or lower in standard in vitro assays that assess endothelin antagonist or agonist activity have pharmacological utility (see, e.g., U.S. Pat. Nos. 5,352,800, 5,334,598, 5,352,659, 5,248,807, 5,240,910, 5,198,548, 5,187,195, and 5,082,838). By virtue of this activity, such compounds are considered to be useful for the treatment of hypertension such as peripheral circulatory failure, heart disease such as angina pectoris, cardiomyopathy, arteriosclerosis, myocardial infarction, pulmonary hypertension, vasospasm, vascular restenosis, Raynaud's disease, cerebral stroke such as cerebral arterial spasm, cerebral ischemia, late phase cerebral spasm after subarachnoid hemorrhage, and other diseases in which endothelin has been implicated.

Accordingly, there is a need for development of new compositions for coating medical devices, including stents and synthetic grafts, to prevent smooth muscle cell proliferation, thereby preventing restenosis. The devices preferably would inhibit local thrombosis without the risk of systemic bleeding complications, provide continuous prevention of arterial injury including local inflammation, and provide sustained prevention of smooth muscle cell proliferation at the site of angioplasty without serious systemic complications. Inasmuch as stents prevent at least a portion of the restenosis process, an agent which prevents inflammation and the proliferation of smooth muscle cells combined with a stent may provide the most efficacious treatment for post-angioplasty restenosis.

### SUMMARY OF THE INVENTION

The present invention meets the above-described needs by providing improved devices and methods for inhibiting restenosis and hyperplasia, particularly after intravascular intervention. In particular, one aspect of the present invention provides medical devices which allow for controlled endothelin (A) receptor antagonist delivery with increased efficiency and/or efficacy to selected locations within a patient's vasculature to inhibit restenosis. Moreover, the present invention provides minimal to no hindrance to endothelialization of the vessel wall.

More specifically, one aspect of this invention provides an implantable medical device comprising a body, a biocompatible matrix coating, and at least one type of an endothelin (A) receptor antagonist in an amount effective to reduce or prevent smooth muscle cell proliferation. In one embodiment, the device comprises an expansible structure which is implantable within a body lumen and means on or within the structure for releasing an endothelin (A) receptor antagonist at a rate selected to minimize and/or inhibit smooth muscle cell proliferation. The endothelin (A) receptor antagonist may be, for example, Ambrisentan.

The expansible structure may be in the form of a stent, which additionally maintains luminal patency, or may be in the form of a graft, which additionally protects or enhances the strength of a luminal wall. The expansible structure may be balloon expandable or self-expanding and is preferably suitable for luminal placement in a body lumen. The body lumen includes any blood vessel in the patient's vasculature, including veins, arteries, aorta, and particularly including coronary and peripheral arteries, as well as previously implanted grafts, shunts, fistulas, and the like. It will be appreciated that the present invention may also be applied to other body lumens, such as the biliary duct, which are subject to excessive neoplastic cell growth, as well as to many internal corporeal tissues, such as organs, nerves, glands, ducts, and the like.

In a first embodiment, the means for releasing an endothelin (A) receptor antagonist comprises a matrix formed over at least a portion of the structure. The matrix may be composed of a material which is degradable, partially degradable, or nondegradable polymer, and which is either a synthetic or natural material. The endothelin (A) receptor antagonist may be disposed within the matrix in a manner that provides the desired release rate. Alternatively, the endothelin (A) receptor antagonist may be disposed on or within the expansible structure and subsequently coated with the matrix to provide the desired release rate.

In some instances, the matrix may comprise multiple adjacent layers of the same or different matrix material, wherein at least one layer contains an endothelin (A) receptor antagonist and another layer contains an endothelin (A) receptor antagonist, at least one substance other than an endothelin (A) receptor antagonist, or no substance. For example, an endothelin (A) receptor antagonist disposed within a top degradable layer of the matrix is released as the top matrix layer degrades and a second substance disposed within an adjacent nondegradable matrix layer is released by diffusion. In some instances, multiple substances may be disposed within a single matrix layer.

In yet another embodiment, the means for releasing the endothelin (A) receptor antagonist comprises channels or micropores on or within the structure containing the endothelin (A) receptor antagonist, and a matrix disposed over the channels or micropores. The matrix may be degradable or partially degradable over a preselected time period so as to provide the desired endothelin (A) receptor antagonist release rate.

Yet another medical device comprises an expansible structure or body, a source of an endothelin (A) receptor antagonist on or within the structure, and a source of at least one other active drug in addition to the endothelin (A) receptor antagonist on or within the structure. The endothelin (A) receptor antagonist and the active drug are released from the source at a desired rate after the expansible structure is implanted in a bodily lumen.

In another aspect of the present invention, methods for inhibiting restenosis in a blood vessel following vascular intervention of the blood vessel are provided. For example, one method may include implanting a vascular device of this invention in the body lumen to prevent reclosure of the blood vessel. An endothelin (A) receptor antagonist is released from the implanted device at a rate selected to inhibit smooth muscle cell proliferation. The device may further release at least one other substance in addition to the endothelin (A) receptor antagonist.

With the devices of this invention, the endothelin (A) receptor antagonist is provided to a bodily lumen in an amount effective to prevent or attenuate smooth muscle cell proliferation within the lumen and, in cases where the device is implanted in a blood vessel, mediate vasodilatation of the vessel into which it is placed. Since placement of an implantable device into a vessel causes endothelial damage leading to elevated endothelin levels and endothelin-mediated smooth muscle cell proliferation, concomitant release of an endothelin (A) receptor antagonist from that device can mitigate the prothrombic and proinflammatory activity of nonquiescent endothelium and prevent intimal proliferation. This inhibition of intimal smooth muscle cells and stroma produced by the smooth muscle allows for more rapid and complete re-endothelization following the intraventional placement of the device with reduced vascular injury.

A device of this invention can be used for any endothelin-mediated condition, e.g., an indication which involves the presence of an activated endothelium that secretes unwanted amounts of endothelin. In many cases, local delivery of endothelin (A) receptor antagonists according to this invention will provide better efficacy than systemic administration.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### BRIEF DESCRIPTION OF THE FIGURES

The accompanying drawings, which are incorporated in and form a part of the specification, illustrate non-limiting embodiments of the present invention, and together with the description serve to explain the principles of the invention.

In the Figures:

FIG. 1 is a three dimensional side perspective view of the self-expanding stent comprising a lattice.

FIG. 2 is a three-dimensional view of one embodiment of a balloon expandable stent according to this invention.

### DETAILED DESCRIPTION OF THE INVENTION

Overview

This invention provides improved prophylactic or therapeutic devices that modulate endothelin-mediated activities. More specifically, one aspect of this invention provides medical devices for maintaining the patency of bodily lumen by preventing smooth muscle cell proliferation within the lumen.

As used herein, “medical device” refers to a device that is introduced temporarily or permanently into a mammal for the prophylaxis or therapy of a medical condition. These devices include any that are introduced subcutaneously, percutaneously or surgically to rest within an organ, tissue or lumen. Medical devices may include stents, synthetic grafts, artificial heart valves, artificial hearts and fixtures to connect the prosthetic organ to the vascular circulation, venous valves, abdominal aortic aneurysm (AAA) grafts, inferior venal caval filters, catheters including permanent drug infusion catheters, embolic coils, embolic materials used in vascular embolization (e.g., PVA foams), and vascular sutures.

The invention has particular application to the stenting of human blood vessels, particularly in the prophylaxis or treatment of restenosis, and will be described in reference thereto for ease of discussion. However, in a broader sense it relates to the stenting of any body passage or lumen. Subjects that can be treated using the methods and devices of this invention include mammals such as a human.

As stated, one cause for restenosis is the proliferation of smooth muscle cells at the site of a lesion such as that caused by angioplasty. Accordingly, it is desirable to prevent or limit accumulation of these fast-replicating smooth muscle cells in an effort to prevent restenosis. The devices of this invention achieve this by providing a local source of an endothelin (A) receptor antagonist that minimizes or prevents smooth muscle cell proliferation. The endothelin (A) receptor antagonist is “provided” by the medical device in that the endothelin (A) receptor antagonist is (i) coated onto the surface of the device as a coating per se or as an integral part of a coating; (ii) compounded into the device material; (iii) bound to a matrix coating of a device; or (iv) held within channels, reservoirs or micropores in the device or delivered through the pores of a porous device, such as in an infusion style catheter such as a channel balloon catheter. The medical device is positioned within a desired target location within the body, whereupon the endothelin (A) receptor antagonist diffuses through or out of the device.

The devices of this invention provide a therapeutically effective amount of an endothelin (A) receptor antagonist to a targeted site such as a diseased or injured bodily lumen. The body lumen may be any blood vessel in the patient's vasculature, including veins, arteries, aorta, and particularly including coronary and peripheral arteries, as well as previously implanted grafts, shunts, fistulas, and the like. It will be appreciated that the present invention may also find use in body lumens other than blood vessels. For example, the present invention may be applied to many internal corporeal tissues, such as organs, nerves, glands, ducts, and the like.

As used herein, a “therapeutically effective amount of the endothelin antagonist” means the amount of an endothelin antagonist that can reduce the sequelae to vascular injury, mitigate the prothrombotic and proinflammatory activity of a non-quiescent endothelium, and/or prevent restenosis following vascular intervention. The precise desired therapeutic effect will vary according to the condition to be treated, the formulation to be administered, and a variety of other factors that are appreciated by those of ordinary skill in the art. The amount of an endothelin antagonist needed to practice the claimed invention also varies with the nature of the endothelin antagonist used. It is well known to those of ordinary skill in the art how to determine therapeutically effective amounts of an endothelin antagonist required to inhibit smooth muscle cell proliferation. An example includes determining the effectiveness of an endothelin (A) receptor antagonist in ameliorating intimal hyperplasia (IH) in cultured human saphenous vein as described by Porter. et al. (J. Vasc. Surg. (1998), 28:695-701) and in Example 3. The effective concentration of endothelin (A) receptor antagonist depends in part on the level and extent of vessel injury, and will typically be in a range from about 0.1 nM to 10 μM.

In one embodiment, the present invention is a stent or catheter for performing or facilitating a medical procedure. Accordingly, the present invention may be used in conjunction with any suitable or desired set of stent components and accessories, and it encompasses any of a multitude of stent designs. These stent designs may include for example a basic solid or tubular flexible stent member or a balloon catheter stent, up to complex devices including multiple tubes or multiple extruded lumens, as well as various accessories such as guidewires, probes, ultrasound, optic fiber, electrophysiology, blood pressure or chemical sampling components. In other words, the present invention may be used in conjunction with any suitable stent or catheter design, and is not limited to a particular type of stent or catheter.

The present invention further provides a method of treating or preventing restenosis in a mammal, comprising inserting a medical device into said vessel, wherein the medical device comprises a hollow body, a matrix coating, and at least one type of an endothelin (A) receptor antagonist in an amount effective to reduce or inhibit smooth muscle cell proliferation. The method can be performed, for example, after angioplasty, wherein the presence of an endothelin antagonist on the medical device further reduces the occurrence of restenosis and thrombosis after medical device implantation into a vessel.

This invention also relates in a broader sense to the prevention of smooth muscle cell proliferation in vivo which is caused by any endothelin-mediated condition. As used herein, an “endothelin-mediated condition” is a condition that is caused by abnormal endothelin activity or one in which compounds that inhibit endothelin activity have therapeutic use. Such conditions include, but are not limited to, cardiovascular disease, restenosis and thrombosis after medical device implantation into a vessel, asthma, inflammatory diseases, ophthalmologic disease, menstrual disorders, obstetric conditions, gastroenteric disease, renal failure, pulmonary hypertension, endotoxin shock, anaphylactic shock, or hemorrhagic shock. Endothelin-mediated conditions also include conditions that result from therapy with agents, such as erythropoietin and immunosuppressants, which elevate endothelin levels.

Stents

In one embodiment, the medical device of this invention is a stent. The term “stent” as used herein means any tubular medical device for insertion into canals, vessels, passageways, cavities, tissues, or organs of a living mammal, which when inserted into the lumen of a vessel expands the cross-sectional lumen of a vessel and serves to keep the passage open. Included are stents that are delivered percutaneously to treat coronary artery occlusions or to seal dissections or aneurysms of the splenic, renal, carotid, iliac and popliteal vessels. In another embodiment, the stent is delivered into a venous vessel.

The underlying structure of the stent can be virtually any stent design. There are typically two types of stents: self-expanding stents and balloon expandable stents. Stents are typically formed from malleable metals, such as 300 series stainless steel, or from resilient metals, such as super-elastic and shape memory alloys, e.g., Nitinol™ alloys, spring stainless steels, and the like. They can also, however, be formed from non-metal materials such as non-degradable or biodegradable polymers or from bioresorbable materials such as levorotatory polylactic acid (L-PLA), polyglycolic acid (PGA) or other materials such as those described in U.S. Pat. No. 6,660, 827.

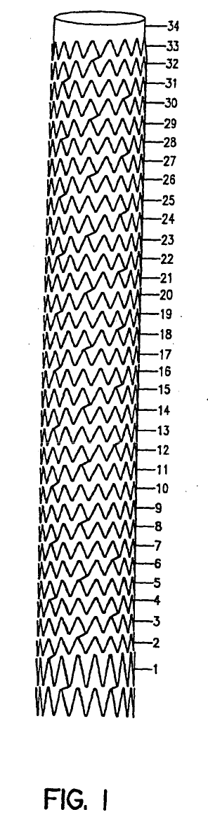

Self-expanding stents are delivered through the body lumen on a catheter to the treatment site where the stent is released from the catheter, allowing the stent to automatically expand and come into direct contact with the luminal wall of the vessel. Examples of self-expanding stent suitable for purposes of this invention are disclosed in U.S. Publication No. 2002/0116044, which is incorporated herein by reference. For example, the self-expanding stent described in U.S. Publication No. 2002/0116044 and illustrated in FIG. 1 comprises a lattice having two different types of helices (labeled 1-33 in FIG. 1) forming a hollow tube having no free ends. The first type of helix is formed from a plurality of undulations, and the second type of helix is formed from a plurality of connection elements in series with the undulations, wherein the connection elements connect fewer than all of the undulations in adjacent turns of the first type of helix. The first and second types of helices proceed circumferentially in opposite directions along the longitudinal axis of the hollow tube. This design provides a stent having a high degree of flexibility as well as radial strength. It will be apparent to those skilled in the art that other self-expanding stent designs (such as resilient metal stent designs) could be used according to this invention.

The stent may also be a balloon expandable stent which is expanded using an inflatable balloon catheter. Balloon expandable stents may be implanted by mounting the stent in an unexpanded or crimped state on a balloon segment of a catheter. The catheter, after having the crimped stent placed thereon, is inserted through a puncture in a vessel wall and moved through the vessel until it is positioned in the portion of the vessel that is in need of repair. The stent is then expanded by inflating the balloon catheter against the inside wall of the vessel. Specifically, the stent is plastically deformed by inflating the balloon so that the diameter of the stent is increased and remains at an increased state, as described in U.S. Pat. No. 6,500,248 B1, which is incorporated herein by reference.

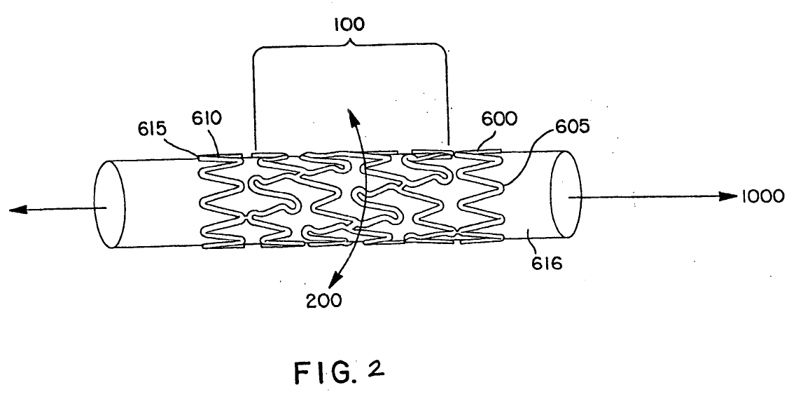

A preferred balloon expandable stent design is that disclosed in U.S. Publication No. 2002/0111669 A1 (incorporated herein by reference) and illustrated in FIG. 2, wherein stent 20 has a geometry that allows it to be readily crimped onto a balloon delivery device. The stent is comprised of a main body 100 mounted on a carrier 616. The main body 100 is comprised of a plurality of first helical segments having a first helical angle α with respect to the longitudinal axis of the stent and a plurality of second helical segments having a second helical angle θ with respect to the longitudinal axis. The helical segments are capable of expanding and contracting circumferentially, i.e., they expand or contract along the circumference of the stent. When the stent is crimped, at least one portion of one first helical segment, along with at least one portion of a second first helical element, nestle between the same two portions of two separate second helical segments. A stent as shown in FIG. 2 thus has a geometry that is well suited for crimping the stent onto a delivery device.

In another embodiment, the stent can be designed to contain channels or micropores that can be loaded with the endothelin (A) receptor antagonist, as described in U.S. Pat. No. 6,273,913 B1, which is incorporated herein by reference. A coating or membrane of biocompatible material could be applied over the channels to control the diffusion of the endothelin (A) receptor antagonist from the reservoirs to the artery wall. In this embodiment the stent is dipped into a solution of endothelin (A) receptor antagonist dissolved in a suitable solvent for sufficient time to allow the solution to permeate into the pores or channels. The dipping solution can also be compressed to improve the loading efficiency. After solvent has been allowed to evaporate, the stent can be dipped briefly in fresh solvent to remove excess surface-bound endothelin (A) receptor antagonist. A matrix solution such as a polymer can be applied to the stent as detailed below. This outer matrix layer will act as diffusion-controller for release of the endothelin (A) receptor antagonist. One advantage of this system is that the properties of the coating can be optimized for achieving superior biocompatibility and adhesion properties, without the additional requirement of being able to load and release the drug. The size, shape, position, and number of channels can be used to control the amount of endothelin (A) receptor antagonist, and therefore the dose delivered.

An exemplary method for preparing a porous stent utilizes the method disclosed in U.S. Pat. No. 5,972,027, which is incorporated herein by reference. Briefly, the method comprises providing a powdered metal or polymeric material, subjecting the powder to high pressure to form a compact, sintering the compact to form a final porous metal or polymer, forming a stent from the porous metal and, optionally, loading at least an endothelin (A) receptor antagonist (and optionally one or more additional drugs) into the pores. The stent may be impregnated with the endothelin (A) receptor antagonist and optionally one or more additional drugs by any known process in the art, including high pressure loading in which the stent is placed in a bath of the desired endothelin (A) receptor antagonist and subjected to high pressure or, alternatively, subjected to a vacuum. Alternatively, rather than loading the porous stent with the endothelin (A) receptor antagonist, the stent is instead implanted in the desired bodily location, and then the endothelin (A) receptor antagonist is injected through a delivery tubing to the hollow stent and then out the pores in the stent to the desired location.

The stent can be made of virtually any biocompatible material having physical properties suitable for the design, and can be biodegradable or nonbiodegradable. The material can be either elastic or inelastic, depending upon the flexibility or elasticity of the polymer layers to be applied over it. Accordingly, the stents of this invention can be prepared in general from a variety of materials including ordinary metals, shape memory alloys, various plastics and polymers, carbons or carbon fibers, cellulose acetate, cellulose nitrate, silicone and the like.

Exemplary biocompatible metals for fabricating the expandable stent include high grade stainless steel, titanium alloys including NiTi (a nickel-titanium based alloy referred to as Nitinol™), cobalt alloys including cobalt-chromium-nickel alloys such as Elgiloy® and Phynox®, niobium-titanium (NbTi) based alloys, tantalum, gold, and platinum-iridium.

Suitable nonmetallic biocompatible materials include, but are not limited to, polyamides, polyolefins (e.g., polypropylene, polyethylene etc.), nonabsorbable polyesters (i.e. polyethylene terephthalate), and bioabsorbable aliphatic polyesters (e.g., homopolymers and copolymers of lactic acid, glycolic acid, lactide, glycolide, para-dioxanone, trimethylene carbonate, ε-caprolactone, etc. and blends thereof).

Synthetic Graft

In another embodiment, the medical device of this invention is a synthetic graft. Grafts, including stent grafts that are provided with a polymeric material/endothelin (A) receptor antagonist matrix in accordance with the present invention, include synthetic vascular grafts that can be used for replacement of blood vessels in part or in whole. Synthetic grafts can be used for end-to-end anastomosis of blood vessels or for bypass of a diseased vessel segment. A typical vascular graft is a synthetic tube, wherein each end thereof is sutured to the remaining ends of a blood vessel from which a diseased or otherwise damaged portion has been removed.

A synthetic graft is typically tubular and may be, e.g., of a woven, knit or velour construction. The vascular grafts may be reinforced with, for example, helices, rings, etc. in order to provide uniform strength over the entire surface of the graft tubing. The materials of which such grafts are constructed are biologically compatible materials including, but not limited to, thermoplastic materials such as polyester, polytetrafluoroethylene (PTFE), polyethylene terephthalate, silicone and polyurethanes. In another embodiment, a synthetic graft is composed of an inner layer of meshed polycarbonate urethane and an outer layer of meshed Dacron. It will be apparent to those skilled in the art that any biocompatible synthetic graft can be used with the endothelin (A) receptor antagonists and matrices of this invention.

It is known to those of ordinary skill in the art that peripheral vessels that are used for vascular grafts in other peripheral sites or in coronary artery bypass grafts, frequently fail due to post surgical stenosis. The synthetic grafts of this invention comprising an endothelin (A) receptor antagonist alleviate this problem in that the graft maintains the vascular luminal. area in surgically traumatized vessels and elution of the endothelin (A) receptor antagonist from the graft retards the ability of the vessel to contract, resulting in a larger luminal diameter or cross-sectional area. Furthermore, the elution of endothelin (A) receptor antagonist can advantageously prevent the constriction or spasm that frequently occurs after vascular grafts are anastomosed to both their proximal and distal locations, which can lead to impaired function, if not total failure, of vascular grafts. Thus, the presence of endothelin (A) receptor antagonist should decrease the incidence of spasms, which can occur from a few days to several months following the graft procedure.

Matrix

A medical device of this invention preferably comprises a matrix which may be prepared from a variety of degradable, partially degradable, nondegradable polymer, synthetic, or natural materials. The endothelin (A) receptor antagonist(s) may be dispersed throughout the matrix material, which is subsequently coated onto the body of the device in a manner that provides the desired release rate of the endothelin (A) receptor antagonist. Alternatively, the endothelin (A) receptor antagonist is disposed on the outer layer of a matrix-coated device. In a further embodiment, the endothelin (A) receptor antagonist is disposed on or within the device structure which is then coated with a matrix that will provide the desired release rate.

The polymer matrix may degrade in vivo by bulk degradation, in which the matrix degrades throughout, or by surface degradation, in which a surface of the matrix degrades over time while maintaining bulk integrity. Hydrophobic matrices are preferred as they are more likely to release an endothelin (A) receptor antagonist at the desired release rate. Alternatively, a nondegradable matrix may release the substance by diffusion. A primary requirement for the matrix is that it be sufficiently elastic and flexible to remain intact upon expansion and durable enough to prevent delamination during deployment of the device.

(A) Naturally Occurring Materials

The matrix may be selected from naturally occurring substances such as film-forming polymeric biomolecules that may be enzymatically degraded in the human body or are hydrolytically unstable in the human body including, but not limited to, fibrin, fibrinogen, heparin, collagen, elastin, fatty acids (and esters thereof), hyaluronic acid, carbon, laminin, and cellulose, and absorbable biocompatable polysaccharides such as chitosan, starch and glucosoamino-glycans.

(B) Fullerenes

The matrix may also comprise a carbon-cage molecule known as fullerene as described in U.S. Pat. No. 6,468,244, which is specifically incorporated herein. Fullerenes may be deposited on surfaces in a variety of different ways, including, sublimation, laser vaporization, sputtering, ion beam, spray coating, dip coating, roll-on or brush coating as disclosed in U.S. Pat. No. 5,558,903, which is specifically incorporated herein. The fullerene surface may be chemically modified to present specifically reactive groups to the endothelin antagonist, e.g., oxidants or reductants. Fullerenes may also form nanotubes that incorporate other atoms or molecules (Liu et al. Science 280:1253-1256 (1998), incorporated herein by reference). The synthesis and preparation of carbon nanotubes is well known in the art (U.S. Pat. No. 5,753,088 to Olk, et al., and U.S. Pat. No. 5,641,466 to Ebbsen, et al., both incorporated herein by reference).

The fullerenes can also be adapted to have a therapeutic effect, or to otherwise perform or enhance a medical procedure. The therapeutic effect may include the generation of oxygen radicals or other reactive oxygen species, especially when the fullerenes are exposed to light or some other kind of activating energy. For example, when activated by light (e.g., by an optical fiber inserted through the hemostatic valve and the inner lumen) the fullerenes may tend to generate oxygen radicals or other reactive oxygen species, or to otherwise have a therapeutic effect. This effect may include inhibiting the proliferation of cells, including smooth muscle cells, to resist restenosis (see U.S. Pat. No. 6,468,244).

(C) Synthetic Materials

The matrix that is used to coat the stent or synthetic graft may also be selected from any biocompatible polymeric material capable of holding the endothelin (A) receptor antagonist. The polymer may be either a biostable or a bioabsorbable polymer depending on the desired rate of release of the endothelin (A) receptor antagonist or the desired degree of polymer stability.

Suitable materials for preparing a polymer matrix include, but are not limited to, polycarboxylic acids, cellulosic polymers, silicone adhesives, fibrin, gelatin, polyvinylpyrrolidones, maleic anhydride polymers, polyamides (e.g., Nylon 66 and polycaprolactam), polyvinyl alcohols, polyethylene glycols, polyethylene oxides, glycosaminoglycans, polysaccharides, polyesters, poly(amino acids)polyurethanes, segmented polyurethane-urea/heparin, silicons, silicone modified (segmented) polyether polyurethane, silicone modified (segmented) polycarbonate polyurethane, silane or silanated polymers, polyorthoesters, polyanhydrides, polycarbonates, polypropylenes, poly-L-lactic acids, polyglycolic acids, polycaprolactones, polyhydroxybutyrate valerates, polyacrylamides, polyethers, polyalkylenes oxalates, poly(iminocarbonates), polyoxaesters, polyamidoesters, polyphosphazenes, vinyl halide polymers, polyvinylidene halides, polyacrylonitrile, polyvinyl ketones, polyvinyl aromatics (e.g., polystyrene), etheylene-methyl methacrylate copolymers, acrylonitrile-styrene copolymers, ABS resins, ethylene-vinyl acetate copolymers, alkyl resins; polycarbonates, polyoxymethylenes, polyimides, epoxy resins, polyurethanes, rayon, rayon-triacetate, cellulose, cellulose acetate, cellulose acetate butyrate, cellophane, cellulose nitrate, cellulose propionate, cellulose ethers (i.e. carboxymethyl cellulose and hydoxyalkyl celluloses) and mixtures and copolymers thereof.

Exemplary biodegradable or bioerodible matrix materials include polyanhydrides, polyorthoesters, polycaprolactone, poly vinly acetate, polyethyl vinyl acetate copolymers, polyhydroxybutyrate-polyhyroxyvalerate, polyglycolic acid, polyactic/polyglycolic acid copolymers and other aliphatic polyesters, aliphatic and hydroxy polymers of lactic acid, glycolic acid, mixed polymers and blends, polyhydroxybutyrates and polyhydroxy-valeriates and corresponding blends, or polydioxanon, modified starch, gelatin, modified cellulose, caprolactone polymers, polyacrylic acid, polymethacrylic acid or derivatives thereof, among a wide variety of polymeric substrates employed for this purpose. Such biodegradable polymers will disintegrate in a controlled manner (depending on the characteristics of the carrier material and the thickness of the layer(s) thereof), with consequent slow release of the endothelin (A) receptor antagonist incorporated therein, while in contact with blood or other body fluids. A discussion of biodegradable coatings is provided in U.S. Pat. No. 5,788,979, which is specifically incorporated herein by reference.

Suitable nondegradable matrix materials include polyurethane, polyethylene imine, cellulose acetate butyrate, ethylene vinyl alcohol copolymer, or the like.

The polymers used for coatings are preferably film-forming polymers that have molecular weight high enough as to not be waxy or tacky. The polymers also preferably adhere to the stent and are not so readily deformable after deposition on the stent as to be able to be displaced by hemodynamic stresses or by friction on the device during deployment. The polymers provide sufficient toughness so that the polymers will not be rubbed off during handling or deployment of the stent and will not crack during expansion of the stent.

In another embodiment, the matrix coating can include an endothelin (A) receptor antagonist and a blend of a first co-polymer having a first, high release rate and a second co-polymer having a second, lower release rate relative to the first release rate as described in U.S. Pat. No. 6,569,195 B2, which is incorporated herein by reference. The first and second copolymers are preferably erodible or biodegradable. In one embodiment, the first copolymer is more hydrophilic than the second copolymer. For example, the first copolymer can include a polylactic acid/polyethylene oxide (PLA-PEO) copolymer and the second copolymer can include a polylactic acid/polycaprolactone (PLA-PCL) copolymer. Formation of PLA-PEO and PLA-PCL copolymers is well known to those skilled in the art. The relative amounts and dosage rates of endothelin (A) receptor antagonist delivered over time can be controlled by controlling the relative amounts of the faster releasing polymer relative to the slower releasing polymer. For higher initial release rates the proportion of faster releasing polymer can be increased relative to the slower releasing polymer. If it is desired to have most of the dosage released over a long time period, the majority of the matrix can be the slower releasing polymer.

In other instances, polymers with different solubilities in solvents can be used to build up different polymer layers that may be used to deliver the endothelin (A) receptor antagonist or control the release profile of the endothelin (A) receptor antagonist. For example since ε-caprolactone-co-lactide elastomers are soluble in ethyl acetate and ε-caprolactone-co-glycolide elastomers are not soluble in ethyl acetate. A first layer of ε-caprolactone-co-glycolide elastomer containing a drug can be over coated with ε-caprolactone-co-glycolide elastomer using a coating solution made with ethyl acetate as the solvent. As will be readily appreciated by those skilled in the art numerous layering approaches can be used to provide the desired delivery rate of the endothelin (A) receptor antagonist.

Addition of Endothelin Antagonist to the Matrix

Endothelin (A) receptor antagonists can be incorporated into the matrix, either covalently or noncovalently, wherein the matrix provides for the controlled release of the endothelin (A) receptor antagonist. In certain cases the endothelin (A) receptor antagonist is incorporated into the matrix by mixing the endothelin antagonist with a matrix material dissolved in an appropriate solvent. Alternatively, the endothelin antagonist may be covalently or noncovalently coated onto the last layer of matrix that is applied to the medical device.

The matrix can be formulated by mixing the endothelin receptor (A) antagonist and optionally one or more additional therapeutic agents with the matrix material in suitable solvent. The endothelin receptor (A) antagonist and the therapeutic agent may be provided as a liquid, a finely divided solid, or any other appropriate physical form. Optionally, the mixture may include one or more additives, e.g., nontoxic auxiliary substances such as diluents, carriers, excipients, stabilizers or the like. Other suitable additives may be formulated with the polymer and endothelin receptor (A) antagonist and pharmaceutically active agent or compound. For example hydrophilic polymers may be added to a biocompatible hydrophobic coating to modify the release profile (or a hydrophobic polymer may be added to a hydrophilic coating to modify the release profile). One example would be adding a hydrophilic polymer selected from the group consisting of polyethylene oxide, polyvinyl pyrrolidone, polyethylene glycol, carboxylmethyl cellulose, hydroxymethyl cellulose and combination thereof to an aliphatic polyester coating to modify the release profile. Appropriate relative amounts can be determined by monitoring the in vitro and/or in vivo release profiles for the endothelin receptor (A) antagonist and the therapeutic agents.

The ratio of the endothelin receptor (A) antagonist to polymer in the solution will depend on the efficacy of the polymer in securing the endothelin receptor (A) antagonist onto the stent and the desired rate at which the coating releases the endothelin receptor (A) antagonist to the tissue of the blood vessel. More polymer may be needed if it has relatively poor efficacy in retaining the endothelin receptor (A) antagonist on the stent and more polymer may be needed in order to provide an elution matrix that limits the elution of a very soluble endothelin receptor (A) antagonist. A wide ratio of endothelin receptor (A) antagonist to polymer could therefore be appropriate and could range from about 10:1 to about 1:100, and more preferably from about 1:4 to about 1:20.

The solvent is chosen such that there is the proper balance of viscosity, deposition level of the matrix material, solubility of the endothelin (A) receptor antagonist, wetting of the stent and evaporation rate of the solvent to properly coat the stent. In the preferred embodiment, the solvent is chosen such the endothelin (A) receptor antagonist and the matrix material are both soluble in the solvent or are dispersed in the solvent.

The endothelin (A) receptor antagonist only needs to be dispersed throughout the solvent so that it may be either in a true solution with the solvent or dispersed in fine particles in the solvent. Preferable conditions for the coating application are when the matrix material and endothelin (A) receptor antagonist have a common solvent. This provides a wet coating that is a true solution. Less desirable, yet still usable are coatings that contain the endothelin (A) receptor antagonist as a solid dispersion in a solution of the matrix material in solvent. Under the dispersion conditions, care must be taken if a slotted or perforated stent is used to ensure that the particle size of the dispersed pharmaceutical powder, both the primary powder size and its aggregates and agglomerates, is small enough not to cause an irregular coating surface or to clog the slots or perforations of the stent. In cases where a dispersion is applied to the stent and it is desired to improve the smoothness of the coating surface or ensure that all particles of the endothelin (A) receptor antagonist are fully encapsulated in the matrix material, or in cases where it is desirable to slow the release rate of the drug, deposited either from dispersion or solution, a top coat of the same matrix material used to provide sustained release of the endothelin (A) receptor antagonist or another matrix material can be applied that further restricts the diffusion of the drug out of the coating.

It is important to choose a solvent, a matrix material and an endothelin (A) receptor antagonist that are mutually compatible. It is preferably that the solvent is capable of placing the matrix material into solution at the concentration desired in the solution. It is also desirable that the solvent and matrix material chosen do not chemically alter the therapeutic character of the endothelin (A) receptor antagonist.

Application of the Matrix to the Medical Device

In accordance with one embodiment of the present invention, one or more endothelin (A) receptor antagonists are applied as an integral part of a coating on at least a portion of the exterior surface of the stent. In order to provide the coated stent according to this embodiment, a solution which includes a solvent, a matrix material dissolved in the solvent, an endothelin (A) receptor antagonist dispersed in the solvent, and optionally an additional therapeutic agent, is first prepared. The solution is applied to the stent and the solvent is allowed to evaporate, thereby leaving on the stent surface a coating of the matrix material and the endothelin (A) receptor antagonist.

Typically, the solution can be applied to the stent by any suitable means such as, for example, by immersion, spraying, or deposition by plasma or vapor deposition. After each layer is applied, the stent is dried before application of the next layer. In one embodiment, a thin, paint-like matrix coating does not exceed an overall thickness of 100 microns. Whether one chooses application by immersion or application by spraying depends principally on the viscosity and surface tension of the solution, however, a fine spray such as that available from an airbrush can provide a coating with better uniformity and will provide better control over the amount of coating material to be applied to the stent. In either a coating applied by spraying or by immersion, multiple application steps are generally desirable to provide improved coating uniformity and improved control over the amount of endothelin (A) receptor antagonist to be applied to the stent. The amount of endothelin receptor (A) antagonist to be included on the stent can be readily controlled by applying multiple thin coats of the solution while allowing it to dry between coats. The overall coating should be thin enough so that it will not significantly increase the profile of the stent for intravascular delivery by stent.

The adhesion of the coating and the rate at which the endothelin receptor (A) antagonist is delivered can be controlled by the selection of an appropriate bioabsorbable or biostable matrix material and by the ratio of endothelin receptor (A) antagonist to matrix material in the solution. The desired release rate profile of an endothelin (A) receptor antagonist from the device can also be tailored, for example, by varying the thickness of each matrix layer, the radial distribution (layer to layer) of the endothelin (A) receptor antagonist in each layer, the mixing method, the amount of the endothelin (A) receptor antagonist, the combination of different matrix polymer materials at different layers, and the crosslink density of the polymeric material, as discussed below.

When the coating is applied by immersion methods, preferably the method is adapted such that the solution or suspension does not completely fill the interior of the stent or block the orifice. Methods are known in the art to prevent such an occurrence, including adapting the surface tension of the solvent used to prepare the composition, clearing the lumen after immersion, and placement of an inner member such as a mandrel with a diameter smaller than the stent lumen in such a way that a passageway exists between all surfaces of the stent and the inner member, thereby allowing the matrix material to coat the surface of the stent without substantially blocking the passages.

Although the goal is to dry the solvent completely from the coating during processing, it is a great advantage for the solvent to be non-toxic, non-carcinogenic and environmentally benign. Mixed solvent systems can also be used to control viscosity and evaporation rates. In all cases, the solvent must not react with or inactivate the endothelin (A) receptor antagonist or react with the matrix material. Preferred solvents include, but are not limited to, acetone, N-methylpyrrolidone (NMP), dimethyl sulfoxide (DMSO), toluene, xylene, methylene chloride, chloroform, 1,1,2-trichloroethane (TCE), various freons, dioxane, ethyl acetate, tetrahydrofuran (THF), dimethylformamide (DMF), dimethylacetamide (DMAC), water, and buffered saline.

In one embodiment, a stent is coated with a mixture of a pre-polymer, cross-linking agents and the endothelin (A) receptor antagonist, and then subjected to a curing step in which the pre-polymer and crosslinking agents cooperate to produce a cured polymer matrix containing the endothelin (A) receptor antagonist. The curing process involves evaporation of the solvent and the curing and crosslinking of the polymer. Of course, the time and temperature may vary with particular pre-polymers, crosslinkers and type of endothelin (A) receptor antagonist.

In some instances, the matrix may comprise multiple adjacent layers of same or different matrix material, wherein at least one layer contains an endothelin (A) receptor antagonist and another layer contains an endothelin (A) receptor antagonist, at least one substance other than an endothelin (A) receptor antagonist, or no substance. For example, an endothelin (A) receptor antagonist disposed within a top degradable layer of the matrix is released as the top matrix layer degrades and a second substance disposed within an adjacent nondegradable matrix layer is released primarily by diffusion. In some instances, multiple substances may be disposed within a single matrix layer.

Coupling of an Endothelin (A) Receptor Antagonist to a Matrix

In another embodiment, the stent body is first coated with a matrix material, and subsequently a layer of an endothelin (A) receptor antagonist is deposited directly onto at least a portion of the outer matrix layer, thus forming a noncovalently coupled (i.e., bound) endothelin (A) receptor antagonist layer over at least a portion of the matrix. If desired, a porous material can be deposited over the endothelin (A) receptor antagonist layer, wherein the porous material includes a polymer and provides for the controlled release of the endothelin (A) receptor antagonist therethrough and further avoids degradation of the endothelin (A) receptor antagonist. Methods of coating a stent according to this embodiment is disclosed in U.S. Pat. No. 6,299,604, which is specifically incorporated herein by reference.

In yet another embodiment, the endothelin (A) receptor antagonist is covalently bound to the outer matrix layer. In certain instances, the endothelin (A) receptor antagonist molecule contains functional (i.e., reactive) groups that allow the molecule to form a covalent bond with the matrix. In certain other instances, the endothelin (A) receptor antagonist can be covalently coupled to the matrix through the use of hetero- or homobifunctional linker molecules. The use of linker molecules in connection with the present invention typically involves first covalently coupling the linker molecules to the matrix after it is adhered to the stent. After covalent coupling to the matrix, the linker molecules provide the matrix with a number of functionally active groups that can be used to covalently couple one or more types of endothelin (A) receptor antagonists. The linker molecules may be coupled to the matrix directly (i.e., through the carboxyl groups), or through well-known coupling chemistries, such as esterification, amidation, and acylation. For example, the linker molecule could be a polyamine functional polymer such as polyethyleneimine (PEI), polyallylamine (PALLA) or polyethyleneglycol (PEG). A variety of PEG derivatives, e.g., mPEG-succinimidyl propionate or mPEG-N-hydroxysuccinimide, together with protocols for covalent coupling, are commercially available from Shearwater Corporation, Birmingham, Ala.. (See also, Weiner et al., J. Biochem. Biophys. Methods 45:211-219 (2000), incorporated herein by reference). It will be appreciated that the selection of the particular coupling agent may depend on the type of endothelin (A) receptor antagonist used and that such selection may be made without undue experimentation.

Encapsulation technologies can also be used to contain the endothelin (A) receptor antagonist within the matrix and to control delivery of this therapeutic agent to the tissue. For example, nanospheres or microspheres ranging from 100 nM to 500 μM can be formed by encapsulating the endothelin (A) receptor antagonist within one matrix material, and then these coated spheres can be added to another matrix material which is used to coat the device. The endothelin (A) receptor antagonist is then released from the spheres either while still entrapped in the coating, or alternatively the entire sphere can be released into the tissue and the endothelin (A) receptor antagonist is released thereafter. Encapsulation is useful for enhancing the stability of the endothelin (A) receptor antagonist and/or augmenting delivery of the therapeutic agent to its target within the tissue.

In another embodiment, the endothelin (A) receptor antagonist can be attached to fullerene layers that have been deposited directly on the surface of the stent. Cross-linking agents may be covalently attached to the fullerenes. The endothelin (A) receptor antagonist is then attached to the cross-linking agent, which in turn is attached to the stent.

Coating a Stent with an Endothelin (A) Receptor Antagonist

In yet another embodiment, a thin layer of an endothelin antagonist is covalently or noncovalently bonded directly onto the exterior surfaces of the stent. In this embodiment, the stent surface is prepared to molecularly receive the endothelin (A) receptor antagonist according to methods known in the art.

Compounded Stents

In an alternative embodiment of a stent according to the invention, the endothelin (A) receptor antagonist is provided throughout the body of the stent by mixing and compounding an endothelin (A) receptor antagonist directly into the stent polymer melt before forming the stent. For example, the endothelin (A) receptor antagonist can be compounded into materials such as silicone rubber or urethane. The compounded material is then processed by conventional method such as extrusion, transfer molding or casting to form a tubular configuration. The stent resulting from this process benefits by having an endothelin (A) receptor antagonist dispersed throughout the entire stent body. Thus, the endothelin (A) receptor antagonist is present at the outer surface of the stent when the stent is in contact with bodily tissues, organs or fluids and acts to prevent or minimize smooth muscle cell proliferation.

Endothelin (A) Receptor Antagonists

As used herein, the term “endothelin antagonist ” is a compound that inhibits endothelin-stimulated vasoconstriction and contraction and other endothelin-mediated physiological responses. The antagonist may act by interfering with the interaction of the endothelin with an endothelin-specific receptor or by interfering with the physiological response to or bioactivity of an endothelin isopeptide, such as vasoconstriction. Thus, as used herein, an endothelin antagonist interferes with endothelin-stimulated vasoconstriction or other response or interferes with the interaction of an endothelin with an endothelin-specific receptor, in particular an ET(A) receptor, as assessed by assays known to those of skill in the art, such as described by Dashwood et al. (Cardiovascular Research, 43 (1999) 445-456), Huckle et al. (Circulation 103 (2001) 1899-1905), and Modesti et al. (Cardiovasc. Pharmacol. 34 (1999) 333-339).

Many known endothelin (A) receptor antagonists can be utilized according to this invention to inhibit or minimize smooth muscle cell proliferation. Further, the term “endothelin (A) receptor antagonist” as used herein also encompasses agents that may not traditionally be classified as an endothelin (A) receptor antagonist but performs the same function of inhibiting or minimizing smooth muscle cell proliferation. Preferably the endothelin (A) receptor antagonist is one that inhibits smooth muscle cell proliferation with an IC₅₀ in the low nanomolar range.

Examples of endothelin (A) receptor antagonist suitable for purposes of this invention include, but are not limited to, Ambrisentan (LU-208025 (BSF-208027) and LU-302146 (BSF-302146); Abbott), TBC-11251 (J. Med. Chem., 40(11):1690-97 (1997)), BMS-193884 (EP 558,258), BMS-207940 (Pharmaprojects (13.06.97)), BQ-123 (Exp. Opin. Invest. Drugs, B(5):475-487 (1997)), SB-209670 (Exp. Opin. Invest. Drugs, B(5): 475-487 (1997)), SB-217242 (Exp. Opin. Invest. Drugs, B(5): 475-487 (1997)), SB-209598 (Trends in Pharmacol. Sci., 17:177-81 (1996)), TAK-044 (Exp. Opin. Invest. Drugs, B(5): 475-487 (1997)), Bosentan (Trends in Pharmacol. Sci., 18:408-412, (1997)), PD-156707 (J. Med. Chem., 40(7):1063-74, (1997)), L-749329 (Bioorg. Med Chem. Lett., 7(3):275-280, 1997)), L-754142 (Exp. Opin. Invest. Drugs, B(5):475-487 (1997)), ABT-627 (J. Med. Chem., 40(20): 3217-27 (1997)), A-127772 (J. Med. Chem., 39(5):1039-1048 (1996)), A-206377 (213^{th} American Chemical Society National Meeting, San Francisco, Calif., USA, Apr. 13-17, 1997, Poster, MEDI 193), A-182086 (J. Med Chem., 40(20): 3217-27 (1997)), EMD-93246 (211^{th} American Chemical Society National Meeting, New Orleans, USA, 1996, Poster, MEDI 143), EMD-122801 (Bioorg. Med. Chem. Lett., 8(1): 17-22, (1998)), ZD-1611 (Trends in Pharmacol. Sci., 18:408-12 (1997)), AC-610612 (R&D Focus Drug News (18.05.98)), T-0201 (70th Annual Meeting of the Japanese Pharmacological Society, Chiba, Japan, Mar. 22-25 1997, Lecture, O-133), and J-104132 (R&D Focus Drug News (15.12.97)). Each of the above references is specifically incorporated herein by reference. In one preferred embodiment the endothelin (A) receptor antagonist is Ambrisentan.

The medical devices of this invention can include other therapeutic or pharmaceutical agents in addition to the endothelin (A) receptor antagonist. These additional agents, like the endothelin (A) receptor antagonist, can be applied to a stent or incorporated into one or more matrix layers and eluted at a controlled rate. The release rate can be further controlled by varying the ratio of the agent to the polymer in the multiple layers. For example, a higher drug-to-polymer ratio in the outer layers than in the inner layers would result in a higher early dose that would decrease over time.

Examples of additional agents that can be included in the devices of this invention include, but are not limited to, antithrombotics; anticoagulants; thorombolytics; growth factors; growth factor inhibitors; antiproliferative/antimitotic agents including natural products such as vinca alkaloids (i.e., vinblastine, vincristine, and vinorelbine), paclitaxel, epidipodophyllotoxins (i.e., etoposide, teniposide), dactinomycin (actinomycin D) daunorubicin, doxorubicin and idarubicin; anthracyclines (mitoxantrone, bleomycins, plicamycin (mithramycin) and mitomycin); enzymes (L-asparaginase which systemically metabolizes L-asparagine and deprives cells which don't have the capacity to synthesize their own asparagine); antioxidants; agents that inhibit hyperplasia and in particular restenosis; antibiotics; antiproliferative/antimitotic alkylating agents such as nitrogen mustards (mechlorethamine, cyclophosphamide and analogs, melphalan, chlorambucil); ethylenimines and methylmelamines (hexamethylmelamine and thiotepa); alkyl sulfonates-busulfan; nirtosoureas (carmustine (BCNU) and analogs, streptozocin); trazenes-dacarbazinine (DTIC); antiproliferative/antimitotic antimetabolites such as folic acid analogs (methotrexate); pyrimidine analogs (fluorouracil, floxuridine, and cytarabine); purine analogs and related inhibitors (mercaptopurine, thioguanine, pentostatin and 2-chlorodeoxyadenosine{cladribine}); platinum coordination complexes (cisplatin, carboplatin), procarbazine; hydroxyurea; mitotane; aminoglutethimide; hormones (e.g., estrogen); anticoaglants (heparin, synthetic heparin salts and other inhibitors of thrombin); fibrinolytic agents (such as tissue plasminogen activator, streptokinase and urokinase); antiplatelets (e.g., aspirin, dipyridamole, ticlopidine, clopidogrel, abciximab); antimigratory agents; antisecretory agents (e.g., breveldin); antiinflammatories such as adrenocortical steroids (cortisol, cortisone, fludrocortisone, prednisone, prednisolone, 6-α-methylprednisolone, triamcinolone, betamethasone, and dexamethasone); non-steroidal agents (salicylic acid derivatives, i.e., aspirin; para-aminophenol derivatives e.g., acetominophen); indole and indene acetic acids (indomethacin, sulindac, and etodalac); heteroaryl acetic acids (tolmetin, diclofenac, and ketorolac); arylpropionic acids (ibuprofen and derivatives); anthranilic acids (mefenamic acid, and meclofenamic acid); enolic acids (piroxicam, tenoxicam, phenylbutazone, and oxyphenthatrazone); nabumetone; gold compounds (auranofin, aurothioglucose, gold sodium thiomalate); immunosuppressives (e.g., cyclosporine, tacrolimus (FK-506), sirolimus (rapamycin), azathioprine, mycophenolate mofetil); angiogenics (e.g., vascular endothelial growth factor (VEGF), fibroblast growth factor (FGF)); nitric oxide donors; anti-sense oligonucleotides; and combinations thereof.

In certain cases, the endothelin (A) receptor antagonist can be used in combination with other active agents that work synergistically to prevent restenosis and accelerated vascular healing. In particular, an endothelin (A) receptor antagonist can be used together with a technology aimed at accelerating endothelialization, which can help to prevent the inflammatory response of a newly formed endothelium that is activated to secrete endothelin.

Release of Endothelin (A) Receptor Antagonist from Device

As stated, one aspect of this invention provides an implantable medical device comprising an expansible structure and means on or within the structure for releasing an endothelin (A) receptor antagonist at a rate effective to inhibit smooth muscle cell proliferation. In one embodiment, the means for releasing the endothelin (A) receptor antagonist comprises a matrix formed over at least a portion of the device. The matrix can be composed of material that undergoes degradation, and the endothelin (A) receptor antagonist may be disposed within the matrix in a pattern that provides the desired release rates. Alternatively, the matrix may be composed of a nondegradable material which releases the endothelin (A) receptor antagonist by diffusion. Suitable nondegradable matrix materials include polyurethane, polyethylene imine, cellulose acetate butyrate, ethylene vinyl alcohol copolymer, or the like.

The device may comprise multiple matrix layers, each layer containing the endothelin (A) receptor antagonist (alone or in combination with an additional therapeutic agent), a therapeutic agent other than an endothelin (A) receptor antagonist, or no substance. For example, a top layer may contain no substance while a bottom layer contains the endothelin (A) receptor antagonist. As the top layer degrades, the endothelin (A) receptor antagonist delivery rate increases.

In another embodiment, the matrix coating can include an endothelin (A) receptor antagonist and a blend of a first co-polymer having a first, high release rate and a second co-polymer having a second, lower release rate relative to the first release rate as described in U.S. Pat. No. 6,569,195 B2 and as described above.

Alternatively, the endothelin (A) receptor antagonist may be disposed directly on or within the device under the matrix layer(s) to provide the desired release rates. The matrix may degrade by bulk degradation, in which the matrix degrades throughout, or preferably by surface degradation, in which only a surface of the matrix degrades over time while maintaining bulk integrity.

In another embodiment, the means for releasing the endothelin (A) receptor antagonist comprises a reservoir on or within the device containing the endothelin (A) receptor antagonist and a matrix over the reservoir. The matrix can degrade over a preselected time period so that release of the endothelin (A) receptor antagonist from the reservoir begins substantially after the preselected time period. The matrix in this case may comprise a polymer matrix, as described above, which releasably contains the endothelin (A) receptor antagonist within the reservoir. Alternatively, the matrix is nondegradable but is a material that allows the endothelin (A) receptor antagonist to diffuse therethrough into the body lumen.

Uses

A number of medical problems are the result of overexuberant cellular proliferation in tubular body structures. Useful therapeutic applications to which the present invention can be applied include, without limitation, methods for treating, ameliorating, reducing and/or inhibiting any lumen or tissue injury, including those that result in denuding the interior wall of a lumen, namely its endothelial lining, including the lining of a blood vessel, urethra, lung, colon, urethra, biliary tree, esophagus, prostate; fallopian tubes, uterus, vascular graft, or the like. Such injuries result from disease, as in the case of atherosclerosis or urethal hyperplasia (strictures), and/or from mechanical injury from, for example, deployment of an endolumenal stent or a catheter-based device, including balloon angioplasty and related devices.

Vascular therapies that benefit using the methods disclosed herein include, without limitation, cardiomyopathies, cardiac and cerebral strokes, embolisms, aneurysms, atherosclerosis, and peripheral and cardiac ischemias. Delivery of endothelin (A) receptor antagonists competent to inhibit or interfere with smooth muscle cell proliferation using the devices of this invention are particularly useful in treating restenosis.

A similar intimal hyperplasia phenomenon has prevented the adoption of small diameter synthetic vascular grafts for use in coronary artery bypass surgery. Other conditions requiring treatment include the growth or regrowth of tumor tissues on or adjacent to body vessels, ducts and passageways.

A large proportion of end-stage renal disease (ESRD) patients use an implanted synthetic vascular graft to provide vascular access for dialysis treatment. Palder, et al. (Ann. Surg., 202:235-239 (1995)) discussed that these grafts typically fail in 14-19 months with a reported primary occlusion rate of 15-50% at one year. Bethard (Kidney Int., 45:1401-1406 (1994)) demonstrated clinically that, most graft failures result from thrombosis (80-90%); and in turn, the thrombosis is typically caused by a low flow condition, most frequently (>90%) stenosis at the graft/vein anastomosis. The stenosis is the result of an overexuberant cellular proliferation that has been observed following other vascular interventions including angioplasty and synthetic graft placement. It is this failure rate, and the attendant need to repair or replace the vascular access that generate the high costs and hospitalization rates associated with the management of the ESRD patient.

### EXAMPLES

This invention is illustrated in the experimental details section which follows. These sections set forth below the understanding of the invention, but are not intended to, and should not be construed to, limit in any way the invention as set forth in the claims which follow thereafter.

### Example 1

### Endothelin (A) Receptor Antagonist Loaded onto a Vascular Stent

A stainless steel stent is sprayed with a solution of Ambrisentan in a 100% ethanol or methanol solvent. The stent is dried and the ethanol is evaporated leaving the Ambrisentan on a stent surface. A 75:25 PLLA/PCL copolymer (sold commercially by Polysciences) is prepared in 1,4 dioxane (sold commercially by Aldrich Chemicals). The Ambrisentan loaded stent is loaded on a mandrel rotating at 200 rpm and a spray gun (sold commercially by Binks Manufacturing) dispenses the copolymer solution in a fine spray on to the Ambrisentan-coated stent as it rotates for a 10-30 second period. The stent is then placed in an oven at 25-35° C. up to 24 hours to complete evaporation of the solvent.

### Example 2

### Endothelin (A) Receptor Antagonist-Containing Matrix Loaded onto a Vascular Stent

Nitinol™ stents are cleaned by placement in an ultrasonic bath of isopropyl alcohol solution for 10 minutes. The stents are dried and plasma cleaned in a plasma chamber. An ethylene vinyl alcohol copolymer (EVOH) solution is made with EVOR, DMSO and Ambrisentan. The mixture is placed in a warm water shaker bath at 60° C. for 24 hours. The solution is cooled and vortexed. The cleaned stents are dipped in the EVOH solution and then passed over a hot plate, for about 3-5 seconds, with a temperature setting of about 60° C. The coated stents are heated for 6 hours in an air box and then placed in an oven at 60° C. under vacuum condition for 24 hours.

### Example 3

### Human Saphenous Vein Model of Vein Graft Intimal Hyperplasia

Culture method. The use of human saphenous vein model of vein graft intimal hyperplasia, in which paired segments of human long saphenous vein (LSV) are cultured with and without an endothelin (A) receptor antagonist, is described by Porter, et al. (J. Vasc. Surg. (1998), 28:695-701) as follows.

Segments of the LSV are obtained from patients who have undergone arterial bypass grafting the segments are transported to the laboratory in a calcium-free physiologic saline solution and prepared for culture. Briefly, the excess fat and the adventitial tissue are dissected from the vessels, which are then opened longitudinally and cut into 0.5-cm lengths. The vessels are pinned, lumenal surface upmost, with fine minuten pins onto a 500-μm mesh resting on a layer of preformed Sylgard resin (Dow Coming, Seneffe, Belgium) in the bottom of a 60×20-mm glass petri dish. Cultures are maintained in RPMI 1640 medium (Northumbria Biologicals, Cramlington, United Kingdom) and supplemented with 30% fetal calf serum for 14 days at 37° C. in a humidified atmosphere of 5% CO₂ in the air. Consecutive segments are then prepared from each vessel, such that they are equivalent before randomization to the different treatment groups. In each group, 1 segment serves as control, and to the others, the endothelin (A) receptor antagonist compounds are added. The compounds are prepared in a 10% dimethyl sulfoxide solution, and the control veins receive an equivalent volume of vehicle only. The culture medium and the compounds are replaced every 2 to 3 days and, after 14 days, while still pinned in the culture dishes, the segments were fixed overnight in 10% formalin solution, processed, and embedded in paraffin. Transverse sections of 4-μm thickness are double-stained with a combined monoclonal anti-smooth muscle actin and Milller's elastin stain to identify the layers of the vein wall. Mouse anti-human alpha smooth muscle actin antibody is applied at a ratio of 1:400, and diaminobenzidine is used as a final reaction product. After this procedure, a Miller's elastin stain is superimposed.

Measurement of neointimal thickness. The measurement of neointimal thickness is made on transverse sections of each vessel with a computerized image analysis system (Improvision, Coventry, United Kingdom). Thirty measurements are made on each vein evenly distributed across the whole section. The measurements are performed by 2 independent observers, with a high level of agreement (interobserver error).

The foregoing description is considered as illustrative only of the principles of the invention. Further, since numerous modifications and changes will be readily apparent to those skilled in the art, it is not desired to limit the invention to the exact construction and process shown as described above. Accordingly, all suitable modifications and equivalents may be resorted to falling within the scope of the invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1. An implantable medical device comprising a body, a biocompatible matrix coating, and at least one type of an endothelin (A) receptor antagonist in an amount effective to reduce or prevent smooth muscle cell proliferation.

2. The medical device of claim 1, wherein said endothelin (A) receptor antagonist is Ambrisentan, TBC-11251, BMS-193884, BMS-207940, BQ-123, SB-209670, SB-217242, SB-209598, TAK-044, Bosentan, PD-156707, L-749329, L-754142, ABT-627, A-127772, A-206377, A-182086, EMD-93246, EMD-122801, ZD-1611, Ac610612, T-0201, or J-104132.

3. The medical device of claim 2, wherein said endothelin (A) receptor antagonist is Ambrisentan.

4. The medical device of claim 1, wherein said matrix is disposed over at least a portion of said body.

5. The medical device of claim 4, wherein the endothelin (A) receptor antagonist is releasably dispersed throughout said matrix.

6. The medical device of claim 1, wherein the endothelin (A) receptor antagonist is disposed on the body and the matrix is disposed over the endothelin (A) receptor antagonist.

7. The medical device of claim 1, wherein the medical device is a stent.

8. The medical device of claim 7, wherein the stent is a self-expanding stent.

9. The medical device of claim 7, wherein the stent is a balloon expandable stent.

10. The medical device of claim 7, wherein the body of the stent comprises channels or pores for containing the endothelin (A) receptor antagonist.

11. The medical device of claim 1, wherein the body is made of stainless steel, a titanium alloy, a nickel-titanium based alloy, a cobalt alloy, a niobium-titanium (NbTi) based alloy, tantalum, gold, platinum-iridium, plastic, a polymer, carbon, carbon fibers, cellulose acetate, cellulose nitrate or silicone.

12. The medical device of claim 11, wherein said nickel-titanium based alloy is Nitinol.

13. The medical device of claim 7, wherein the body is formed from a bioresorbable material.

14. A stent for reducing or preventing restenosis in an artery, comprising a body, a matrix coating, and Ambrisentan in an amount effective to reduce or prevent smooth muscle cell proliferation.

15. The stent of claim 14, wherein said body is made of Nitinol.

16. A method of treating or preventing restenosis in a bodily vessel, comprising inserting a medical device into said vessel, wherein the medical device comprises a hollow body, a matrix coating, and at least one type of an endothelin (A) receptor antagonist in an amount effective to reduce or inhibit smooth muscle cell proliferation.

17. The method of claim 16 wherein the vessel is a coronary artery.

18. The method of claim 16 wherein the vessel is a peripheral artery.

19. The method of claim 16 , wherein the endothelin receptor (A) antagonist is Ambrisentan, TBC-11251, BMS-193884, BMS-207940, BQ-123, SB-209670, SB-217242, SB-209598, TAK-044, Bosentan, PD-156707, L-749329, L-754142, ABT-627, A-127772, A-206377, A-182086, EMD-93246, EMD-122801, ZD-1611, AC610612, T-0201, or J-104132.

20. The method of claim 19, wherein said endothelin (A) receptor antagonist is Ambrisentan.
